(19) United States
(12) Patent Application Publication
(10) Pub. No.: US 2021/0000091 A1
(43) Pub. Date: Jan. 7, 2021

(54) Split Trans-Complementing Gene-Drive System for Suppressing Aedes Aegypti Mosquitos

(71) Applicant: THE REGENTS OF THE UNIVERSITY OF CALIFORNIA, OAKLAND, CA (US)

(72) Inventors: Ethan Bier, San Diego, CA (US); Valentino Gantz, San Diego, CA (US); Anthony A. James, Irvine, CA (US)

(21) Appl. No.: 16/325,110

(22) PCT Filed: Aug. 17, 2017
(86) PCT No.: PCT/US2017/047304

§ 371 Date: Feb. 12, 2019

Related U.S. Application Data
(60) Provisional application No. 62/375,973, filed on Aug. 17, 2016.

(51) Int. Cl. A01K 67/033; C12N 9/22

(52) U.S. Cl. CPC A01K 67/0339; C12N 9/22

(57) ABSTRACT

Provided are systems, constructs, genetically modified organisms, and methods for greatly reducing or eliminating local populations of Aedes aegypti mosquitoes, and associated Dengue fever, yellow fever, Zika virus, and Chikungunya virus. Provided are genetically modified Aedes aegypti having a Cas9-mediated split gene-drive system for masculinizing the mosquito and ensuring that any female carries a sterile mutation. In addition, gRNAs direct Cas9 cleavage of insecticide-resistance loci, rendering female mosquitoes escaping the male converting gene drive sensitive to insecticides.

### CROSS-REFERENCE TO RELATED APPLICATIONS

This application claims priority to U.S. Provisional Patent Application Ser. No. 62/375,973 filed on Aug. 17, 2016, the entire contents of which are hereby incorporated by reference.

### BACKGROUND

The last decade has seen the global emergence and re-emergence of a number of dangerous mosquito-borne viruses and associated diseases. The distribution and incidence of dengue has increased significantly with 2014 being the worst year world-wide on record, and Hawaii, Puerto Rico and southern Florida experiencing epidemics in the US. Both Chikungunya and Zika viruses were introduced into the western hemisphere and are poised to sweep throughout people who live in the range of the mosquito vectors that transmit them. It is clear that the current suite of vector control strategies, methods developed more than 70 years ago, are no longer adequate to manage the threats of these viruses. Researchers exploiting modern molecular genetics are developing new strategies that show great promise for meeting the challenges of controlling transmission of these pathogens. Recent breakthroughs have now brought these technologies to a stage warranting translation to the field.

The existing art for mosquito control is either to kill them with broadly acting chemical insecticides, or by releasing large numbers of sterile males generated either via radiation or by genetic means.

### SUMMARY OF THE INVENTION

The present disclosure represents a new paradigm for mosquito population suppression. In embodiments, the invention combines split gene drives (facilitating mosquito husbandry and lowering product production costs when compared to Sterile Insect Technology [SIT]), a masculanizing technique (which acts similar to SIT, without lowering fitness due to heavy irradiation) and a female sterile fail-safe mechanism acting as a secondary built-in population suppression strategy.

In embodiments, the present invention combines two concepts: 1) the split or trans-complementing mutagenic chain reaction (MCR) form of gene drive, and 2) the fact that in the mosquito Aedes aegypti sex is determined by the presence of single genetic locus M, present in males but absent in females (Hall et al. 2015). In embodiments, the invention uses the split gene-drive system to transmit a transgene encoding the M-locus (the Nix gene) to all, or nearly all, offspring, thus rendering all such progeny male. The effect of spreading such a paired couple of elements through a population is to convert the entire population to males, which are then unable to procreate. Furthermore, in embodiments, the system renders any female mosquitoes that escaped conversion sterile and/or sensitive to pesticides to which the existing population had acquired resistance.

In embodiments, the invention provides genetically modified strains of Aedes aegypti, and methods for their development, that function in population suppression, population modification, and a combined approach known as reduce and replace (REF). This dual activity of the latter has the advantage of rendering any individual that may escape suppression refractory to the targeted viral pathogens, thereby increasing the prospect of completely interrupting disease transmission. The strains use Cas9-mediated gene-drive to facilitate rapid introgression of the effector elements into target populations without the need of continuous releases of large numbers of mosquitoes. This feature greatly reduces cost and increases sustainability as compared with alternative genetic and Sterile Insect Technologies (SIT) approaches.

In embodiments, the invention provides that two separate genetic elements comprise the split trans-complementing gene-drive suppression system in which the first element (A) carries the male sex-determining locus (M) at a defined autosomal location (e.g., kh locus on third chromosome, or inside the coding region of a gene required for spermatogenesis) such that it can be driven by a Cas9 source provided in trans (element B) (FIG. 1). In the strain carrying element A, the endogenous male-determining locus on the first-chromosome (or X chromosome) is segregated away resulting in transplantation of the M locus to a precisely defined autosomal site. The A element can also carry several guide RNAs (gRNAs): 1) a gRNA driving the element A at its insertion site, 2) a gRNA driving the element B at its insertion site, and 3) multiple gRNAs targeting coding sequences of several genes required for female fertility for mutagenesis through non-homologous end joining (NHEJ). When strains A and B are crossed, however, (males of A crossed to females of B) the Cas9 carried by element B drives copying of both element A and element B at their respective location by means of copying them onto the homologous chromosome, the resulting progeny carrying both elements are male, and capable of transmitting the masculanizing trait on to nearly all their progeny and subsequent generations.

In alternative embodiments, the invention can include the gRNA driving element B along with the Cas9 source to create a full gene drive at the locus (FIG. 2). The advantage of this latter configuration is that it reduces the number of gRNAs needed to be expressed from element A. The advantage of the former trans-complementing MCR configuration is that both strains A and B would be non-driving, simplifying husbandry of these strains prior to crossing them to establish a bipartite gene drive. Elements A and B or the corresponding genomic insertion sites on wild-type chromosomes can also carry fluorescent marker genes to distinguish transgenic from wild-type chromosomes (see legends to FIGS. 1-3).

These male-drives result in the eventual conversion of the entire mosquito population into males. It is possible in certain embodiments that either element does not successfully convert the opposing chromosome at some low frequency. This may arise due to errors in copying of element A (or element B) (e.g., alleles at the M locus generated by non-homologous end joining). These individuals, while still male, can be capable of generating unwanted female progeny. However, the design features are such that any female descendants carry alleles generated by the gRNAs carried on element A that target coding regions of loci required for female fertility. This mechanism ensures that any escaping female carries a sterile mutation so that it does not contribute to the next generation of mosquitoes. In addition, gRNAs can be carried by element A that direct Cas9 cleavage of known loci where insecticide-resistance alleles have been identified, but not cut the wild-type insecticide-sensitive alleles. Such gRNAs can render any potential female mosquitoes escaping the male converting gene drive sensitive to insecticides so that they can be eliminated by standard vector control measures.

Embodiments of the invention are based in part on the high efficiency observed with copying of a single-unit MCR (e.g., one in which the Cas9 source and a gRNA are carried as a single cassette inserted into the site cut by the gRNA=95% conversion efficiency for published y-MCR in fruit flies) and a 99.5% conversion for a MCR carrying a ˜17 kb cargo insert in mosquitoes. In embodiments, the invention provides gene-drive systems that work effectively in Aedes aegypti and that the described split systems effectively drive the masculanizing M-locus to the vast majority of progeny. Therefore, embodiments of the invention provide systems, constructs, genetically modified organisms, and methods for greatly reducing or eliminating local populations of Aedes aegypti mosquitoes and/or rendering mosquitoes sensitive to insecticides, thereby reducing or eradicating Dengue Fever, Zika virus, Chikungunya virus and yellow fever.

### BRIEF DESCRIPTION OF THE DRAWINGS

A better understanding of the features and advantages of the present disclosure will be obtained by reference to the following detailed description that sets forth illustrative embodiments, in which the principles of the disclosure are utilized, and the accompanying drawings of which:

FIG. 1 is a split gene-drive system for masculanizing Aedes aegypti.

FIG. 2 is an alternative embodiment for a full gene-drive system for masculanizing Aedes aegypti.

FIGS. 3A-C show strains for establishing a split trans-complementing gene drive with a combination of two elements and a suppressor strain crossed to wild type females.

### DETAILED DESCRIPTION

All publications, patents, and patent applications mentioned in this specification are herein incorporated by reference to the same extent as if each individual publication, patent, or patent application was specifically and individually indicated to be incorporated by reference.

### Definitions

The term “CopyCat element” refers to a split Cas protein and gRNA configuration, in which only the gRNA can be inserted at the cut site. A CopyCat element can refer to the self-propagating gRNA. The Cas9 source can be supplied in trans, allowing the CopyCat element to be segregated away from the Cas9 source as desired, at which point it will obey the laws of standard Mendelian inheritance. In the presence of Cas9, however, the CopyCat element can be actively copied to its sister chromosome, resulting in it becoming homozygous. An advantage of the CopyCat element is that one can segregate the source of Cas9 away from the CopyCat element and then manipulate such element via standard Mendelian genetics.

The term “effector cassette” can refer to a transgene encoding a protein that when expressed exerts a desired effect (e.g., M factor to confer male sexuality, anti-viral peptides such as anti-viral RNAi, etc.).

The term “endonuclease” refers to an enzyme that cleaves the phosphodiester bond within a polynucleotide chain. Endonucleases can include Cas proteins, such as Cas9.

The term “guide polynucleotide” refers to a polynucleotide sequence that can form a complex with an endonuclease (e.g., Cas protein such as Cas9) and enables the endonuclease to recognize and optionally cleave a target site on a polynucleotide such as DNA. That is, a guide polynucleotide is any polynucleotide sequence having sufficient complementarity with a target polynucleotide sequence to hybridize with the target sequence and direct sequence-specific binding of a CRISPR complex to the target sequence. A guide polynucleotide that solely comprises ribonucleic acids is also referred to as a “guide RNA”.

The term “active genetics” refers to genetic manipulations in which Cas9 and gRNA elements are used to copy a genetic element from one chromosome to the identical insertion site on the sister chromosome and/or actively edit a genome sequence (e.g. sequence deletions, additions) by single-unit MCR or trans-complementing MCR.

The term “genetic drive” can refer to the inheritance of an allele of a diploid gene more than 50% of the time (i.e., more than by random chance alone).

The term “trans-complementing MCR” refers to a configuration in which a gRNA bearing transgene not encoding Cas9 is combined with a Cas9 bearing transgene to actively copy the gRNA bearing transgene to its sister chromosome, actively copy the Cas9 bearing transgene to its sister chromosome, and/or actively edit the genome sequence.

The term “trans-complementing MCR construct,” “transgenic element,” “trans-complementing MCR element,” and the like, refers to a construct that, when co-expressed with at least one other trans-complementing MCR construct, results in trans-complementing MCR. A trans-complementing MCR construct can comprise sequences encoding Cas9, gRNAs, and/or effector cassettes.

### CRISPR/Cas System

The present disclosure is based in part on the CRISPR/Cas system, a genome editing tool that can be used in a wide variety of organisms (e.g., used to add, disrupt, or change the sequence of specific genes). The CRISPR/Cas9 system is based on two elements. The first element, Cas9, is an endonuclease that has a binding site for the second element, which is the guide polynucleotide (e.g., guide RNA). The guide polynucleotide (e.g., guide RNA) directs the Cas9 protein to double stranded DNA templates based on sequence homology. The Cas9 protein then cleaves that DNA template. By delivering the Cas9 protein and appropriate guide polynucleotides (e.g., guide RNAs) into a cell, the organism's genome is cut at a desired location. Following cleavage of a targeted genomic sequence by a Cas9/gRNA complex, one of two alternative DNA repair mechanisms can restore chromosomal integrity: 1) non-homologous end joining (NHEJ) which generates insertions and/or deletions of a few base-pairs (bp) of DNA at the gRNA cut site, or 2) homology-directed repair (HDR) which can correct the lesion via an additional “bridging” DNA template that spans the gRNA cut site. Further aspects of the CRISPR/Cas system known to those of ordinary skill are described in PCT Publication No. WO 2017/049266, the entire contents of which are hereby incorporated by reference.

### Autocatalytic Genome Editing Using Trans-Complementation

The present disclosure provides methods and compositions for autocatalytic genome editing based on genomic integration of split or trans-complementing Mutagenic Chain Reaction (MCR) constructs. Trans-complementing MCR provides a split system, which can consist of two separate transgenic elements which when combined can lead to autocatalytic copying of elements to sister chromosomes and/or active genome sequence editing. One element expresses a Cas9 endonuclease (i.e. the Cas9 bearing element) and the other element (i.e. the non-Cas9 bearing element), which can be inserted elsewhere on the same chromosome as the Cas9-bearing element or on a different chromosome, encodes at least one gRNA that can cut at the site of genomic insertion of the non-Cas9 bearing element (i.e., gRNA1). A second gRNA that cuts at the genomic site of insertion of the Cas9 bearing element can be encoded in either element (i.e., gRNA2). Additional gRNAs such as those that target coding regions of loci required for female fertility and/or insecticide resistance can be encoded in the non-Cas9 bearing element (e.g. gRNA3, etc.). When these two elements are carried in the same individual (e.g., in progeny resulting from a cross of two individuals carrying a respective one of the two elements) both elements are actively copied onto their sister chromosomes and any additional gRNAs cause active genome sequence editing.

In embodiments, a trans-complementing MCR described herein can mitigate problems associated with single-unit MCR since the two separate elements (i.e. cas9 and gRNA1) can each be propagated safely as neither alone can create a gene-drive. Also, neither element alone can create a significant level of off-target mutagenesis since both elements must be combined. Thus, the two separate components of the trans-complementing MCR can be kept separately until the time they are to be used at which point the two stocks can be crossed. The resulting progeny of this cross can then carry both elements which can propagate as a unit like a single-unit MCR.

A trans-complementing MCR can have the same high efficiency observed for a single-unit MCR (e.g., one in which the Cas9 source and a gRNA are carried as a single cassette inserted into the site cut by the gRNA=95% conversion efficiency).

Trans-complementing MCR generally requires at least two trans-complementing constructs, although there could be more. The first trans-complementing construct, which can be referred to as the Cas9 bearing construct, comprises: (1) a DNA fragment encoding an endonuclease (e.g. Cas9 protein) or homolog that directs its expression in the germline cells, and (2) optionally, a sequence encoding a guide polynucleotide (e.g., guide RNA) that can cut at the site of genomic insertion of the first trans-complementing construct (i.e., gRNA2). The second trans-complementing construct, which can be referred to as the non-Cas9 bearing construct, comprises: (1) one or more sequences encoding one or more guide polynucleotides (e.g., guide RNAs); and (2) one or more effector cassettes (e.g., a DNA sequence that carries out a function). The one or more sequences encoding one or more guide polynucleotides in the second trans-complementing construct can include: (1) a sequence encoding a guide polynucleotide that can cut at the site of genomic insertion of the second trans-complementing construct (i.e., gRNA1), (2) optionally, a sequence encoding a guide polynucleotide that can cut at the site of genomic insertion of the first trans-complementing construct (i.e., gRNA2), and (3) optionally one or more sequences encoding guide polynucleotide(s) that can cut at loci required for female fertility and/or insecticide resistance (e.g., gRNA3, gRNA4, gRNA5, etc.). The trans-complementing constructs or proteins encoded therein can also include functional groups, such as for example a GFP domain or other fluoresecent marker, for visualization purposes.

Each of the first and second trans-complementing constructs can be inserted into the genome independently (e.g., by co-injecting a plasmid containing the first trans-complementing construct with a plasmid encoding only the gRNA2 transcript (if needed), and by injecting a plasmid containing the second trans-complementing construct with a plasmid encoding Cas9 or purified Cas9 protein). For example, a plasmid encoded cassette carrying genes encoding the Cas9 protein flanked by homology arms corresponding to the genomic sequences straddling the target site injected with a plasmid encoding only the gRNA2 transcript results in cleavage and homology driven insertion of the sequence encoding the Cas9 protein element into the targeted locus. In another example, a plasmid encoded cassette carrying genes encoding guide RNA(s) targeting genomic sequences of interest and/or an effector cassette, both of which are flanked by homology arms corresponding to the genomic sequences straddling the target site, injected with a plasmid encoding Cas9 or purified Cas9 protein results in cleavage and homology driven insertion of the sequence encoding the guide RNA(s) targeting genomic sequences of interest and/or an effector cassette into the targeted locus.

In embodiments where the sequence encoding the gRNA that cuts at the Cas9 bearing construct site (i.e., gRNA2) is included in the non-Cas9 bearing construct (i.e. the second trans-complementing construct), each of the first and second trans-complementing constructs, if integrated into the genome of germline cells at their respective gRNA sites, can be inherited in a standard Mendelian fashion. When individuals separately carrying these two elements are crossed to each other, the resulting progeny can have both elements and the two elements can propagate like a standard MCR element in that the two parts (i.e., the Cas9 bearing construct inserted at gRNA2's cut-site, and the non-Cas9 bearing construct inserted at gRNA1's cut-site) can copy themselves from one chromosome to the sister chromosome. Because both elements can copy themselves onto the opposing chromosome, these progeny become homozygous for the constructs and all (or nearly all) of the progeny's progeny can inherit the constructs. Also, any additional gRNAs present create homozygous mutations at their respective cut sites by active genome sequence editing and any effector constructs become homozygous as well. The progeny's progeny themselves become homozygous via trans-complementing MCR, and thus can pass on both constructs to their offspring. Thus, trans-complementing MCR generates homozygous mutant phenotypes in a single generation.

In embodiments where the sequence encoding the gRNA that cuts at the Cas9 bearing construct site (i.e., gRNA2) is included in the Cas9 bearing construct (i.e. the first trans-complementing construct), the first trans-complementing construct can always copy itself onto the opposing chromosome and all (or nearly all) progeny from such a parent inherit the first trans-complementing construct. The second trans-complementing construct, however, can be inherited in a standard Mendelian fashion. When individuals separately carrying the first and second trans-complementing constructs are crossed to each other, the resulting progeny can have both constructs and the second trans-complementing construct (i.e. the non-Cas9 bearing construct) can then propagate like a standard MCR element in that the second trans-complementing construct (inserted at gRNA1's cut-site) can copy itself from one chromosome to the sister chromosome. Because both elements can copy themselves onto the opposing chromosome, these progeny become homozygous for the constructs and all (or nearly all) of the progeny's progeny can inherit the constructs. Also, any additional gRNAs present create homozygous mutations at their respective cut sites by active genome sequence editing and any effector constructs become homozygous as well. The progeny's progeny themselves become homozygous via trans-complementing MCR, and thus can pass on both constructs to their offspring. Thus, trans-complementing MCR generates homozygous mutant phenotypes in a single generation.

In embodiments, the disclosure provides methods of independently inserting a first trans-complementing construct into the germline of a first organism (e.g. Aedes Aegypti) and a second trans-complementing construct into the germline of a second organism (e.g. Aedes Aegypti), and obtaining transgenic organisms carrying the insertion of either one of the constructs on one copy of a chromosome. In embodiments, mating between one organism having a first trans-complementing construct and a second organism having a second trans-complementing construct yields progeny containing both constructs, which results in each construct spreading to both chromosomes to create homozygous mutations for each construct by trans-complementing MCR. Any additional gRNAs present create homozygous mutations at their respective cut sites by active genome sequence editing and any effector constructs become homozygous as well. A transgenic organism containing both constructs propagates mutations via the germline to its offspring with greater than 95% efficiency.

In embodiments, trans-complementing MCR can be used to accelerate genetic manipulations and genome engineering. For example, an active trans-complementing MCR drive may provide faster propagation of a genetic trait than passive

Mendelian inheritance. In some embodiments, trans-complementing MCR can selectively add, delete, or mutate genes. In some embodiments, trans-complementing MCR can form a gene drive for spreading genes or exogenous DNA fragments through a population of an organism (e.g. a mosquito) to combat the organism and any diseases/pathogens carried by it (e.g. spreading genes in a mosquito that make all the mosquitoes male or mutating genes to confer infertility or increased susceptibility to pesticides). That is, trans-complementing MCR can be used to disperse (or drive) transgenes into pest populations to selectively inhibit propagation of pest populations and combat propagation of insect borne pathogens or diseases.

In some embodiments, the present disclosure provides trans-complementing MCR drives which offer potential husbandry advantages. In embodiments, there are two separate trans-complementing drives for the cas9<cas9> and gRNAs <gRNA1; gRNA2; gRNA3; effector cassette> wherein gRNA1 cuts at the integration site of the <gRNA1; gRNA2; gRNA3; effector cassette> element and gRNA2 directs cleavage at the site of cas9 genomic insertion. Since neither of the two constructs alone constitutes a drive, each single element can be propagated safely as a separate stock. When the two stocks are crossed (possibly after amplification of each of the stocks for release purposes), a full drive can result. In progeny of this cross, the resulting <cas9> and <gRNA1; gRNA2; gRNA3; effector cassette> can combine to create a drive that can behave thereafter as a linked <cas9; gRNA1; gRNA2; gRNA3; effector cassette> MCR.

In some embodiments, the present disclosure provides alternative trans-complementing MCR drives which offer potential husbandry advantages. In embodiments, there are two separate trans-complementing drives for the cas9<cas9; gRNA2> and gRNAs <gRNA1; gRNA3; effector cassette> wherein gRNA1 cuts at the integration site of the <gRNA1; gRNA3; effector cassette> element and gRNA2 directs cleavage at the site of <cas9; gRNA2> genomic insertion. The <cas9; gRNA2> construct behaves like a full gene drive. However, the <gRNA1; gRNA3; effector cassette> construct alone does not constitute a gene drive and can be propagated safely as a separate stock. When the two stocks are crossed (possibly after amplification of each of the stocks for release purposes) a full drive for both elements can result. In progeny of this cross the resulting <cas9; gRNA2>; <gRNA1; gRNA3; effector cassette> can combine to create a drive that can behave thereafter as a linked <cas9; gRNA1; gRNA2; gRNA3; effector cassette> MCR.

Methods of the disclosure can be used to generate specific strains, breeds, or mutants of an organism; for one-step mutagenesis schemes to generate scoreable recessive mutant phenotypes in a single generation; facilitate basic genetic manipulations in organisms; and accelerate genetic manipulations in organisms.

In embodiments, DNA cuts generated by an endonuclease such as Cas9 may be corrected using different cellular repair mechanisms, including error-prone non-homologous end joining (NHEJ) and Homology Directed Repair (HDR). In some embodiments, a trans-complementing element is integrated into a genome using HDR. Trans-complementing elements are often integrated into a genome using homology directed repair (˜90-100% efficiency).

Trans-complementing elements, when combined, can form an active gene drive and the efficiency of a trans-complementing element integrating into a genome is about 80%, 85%, 90%, 91%, 92%, 93%, 94%, 95%, 96%, 97%, 98%, 99%, 99.5%, 99.9%, or more than 99.9%. In embodiments, the efficiency of allelic conversion of a trans-complementing element in an active gene drive into a genome is about 80%, 85%, 90%, 91%, 92%, 93%, 94%, 95%, 96%, 97%, 98%, 99%, 99.5%, 99.9%, or more than 99.9%. Trans-complementing elements, when combined, form an active gene drive and nearly double their frequency in a population at each generation, as they convert non-MCR chromosomes derived from parents to the MCR condition. This results in a potent gene drive system for spreading genes or exogenous DNA fragments throughout populations of organisms.

In embodiments, a trans-complementing construct is integrated into a defined site on a single copy of a chromosome. For instance, specific targeting via a guide polynucleotide (e.g., guide RNA) directs an endonuclease (e.g., Cas9) to cleave the genome at a specific site, and the trans-complementing construct is inserted into the site by homologous repair. The trans-complementing construct in combination with a supporting trans-complementing construct carry all the elements necessary for insertion of the trans-complementing construct into the same site on a second copy of the chromosome, and combined the trans-complementing constructs cleave the other allele in a cell at the same place as the trans-complementing construct and insert the trans-complementing construct into the second copy of the chromosome thereby resulting in the insertion becoming homozygous. The MCR insertion becomes homozygous in the germline, resulting in progeny of an individual carrying an MCR allele inheriting it. The mutation spreads from a single chromosome to both chromosomes in the next generation to once again become homozygous.

In embodiments, an autocatalytic genetic behavior with self-propagating genetic elements can be achieved in which mutants are generated by two co-expressed trans-complementing constructs that combined encode at least the following two components: (1) a Cas9 protein; and (2) gRNAs targeted to genomic sequences of interest. Such a system can result in Cas9 cutting genomic targets at the sites determined by the gRNAs followed by insertion of a Cas9 bearing element and a non-Cas9 bearing element (e.g. gRNA/effector sequence-bearing element) into the respective loci via HDR. Expression of Cas9 and the gRNAs from the insertion alleles can then lead to cleavage of the opposing alleles followed by HDR-driven insertion of the respective Cas9/gRNA elements into the companion chromosomes.

In embodiments, methods for autocatalytic genome editing in an organism are provided, the methods comprising: (1) integrating a first transgenic element comprising a gene for an endonuclease and optionally a sequence for a guide polynucleotide engineered to target an integration site of the first transgenic element into a first organism; (2) integrating a second transgenic element comprising a sequence for a guide polynucleotide engineered to target an integration site of the second transgenic element, optionally a sequence for a guide polynucleotide engineered to target an integration site of the first transgenic element, one or more sequences for one or more guide polynucleotides engineered to target fertility and/or pesticide resistance loci and cause site directed mutagenesis, and one or more effector cassettes into a second organism; and (3) crossing the first and second organism, wherein crossing the first and second organisms produces progeny that propagates the first transgenic element, the second transgenic element, and site directed mutations to target fertility and/or pesticide resistance loci by mutagenic chain reaction. In embodiments, the organism is an Aedes Aegypti mosquito. In embodiments, the endonuclease is Cas9. In embodiments, the first transgenic element comprises a guide polynucleotide engineered to target an integration site of the first transgenic element. In embodiments, the second transgenic element comprises a guide polynucleotide engineered to target an integration site of the first transgenic element. In embodiments, the effector cassette comprises the male sex-determining locus (M). In embodiments, the effector cassette encodes anti-viral peptides.

Genes involved in female fertility in the vector Aedes aegypti are known to those of ordinary skill and can be targets for site directed mutations by mutagenic chain reaction. Examples of genes involved in female fertility can include AeSCP-2, AeAct-4, AAEL002000, AAEL005747, AAEL005656, AAEL017015, AAEL005212, AAEL005922, AAEL000903, AAEL005049, AAEL007698, and AAEL007823 (US Pub. No. 2017/0071208). Genes involved in insecticide resistance in the vector Aedes aegypti are known to those of ordinary skill and can be targets for site directed mutations by mutagenic chain reaction. Examples of genes involved in insecticide resistance can include CYP9J9, CYP9J10, CYP9J26, CYP9J27, CYP9J28, CYP6Z8, CYP6Z9, CYP9J24, CYP9J26, CYP9J32, GSTE2, GSTE7, CYP9J19, ABCB4, GSTE5, GSTI1, GSTO1, GSTX2, CCae3A, and CYP6N12 (Vontas, J, et al. “Insecticide Resistance In the Major Dengue Vectors Aedes Albopictus and Aedes Aegypti.” Pesticide biochemistry and physiology 104.2 (2012): 126-131).

### EXAMPLES

In embodiments, the present invention provides a genetically modified Aedes aegypti having a Cas9-mediated split gene-drive system for masculinizing the Aedes aegypti. In embodiments, the genetically modified Aedes aegypti further have a Cas9-mediated gene drive system targeting fertility loci and carrying anti-viral effector cassettes. In addition, in embodiments, the present invention provides that gRNAs direct Cas9 cleavage of insecticide-resistance loci, rendering the mosquitoes sensitive to insecticides.

In embodiments, the present invention provides systems, constructs, genetically modified organisms, and methods for reducing or eliminating local populations of Aedes aegypti mosquitoes, and associated Dengue fever, yellow fever, Zika virus, and Chikungunya virus.

### Example 1

Develop population suppression/modification strains of Ae. aegypti based on Cas9-mediated gene drive systems targeting fertility loci and carrying anti-viral effector cassettes.

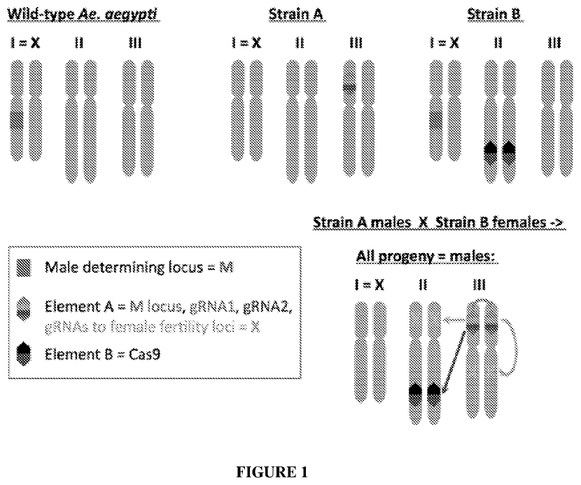

FIG. 1 illustrates a split gene-drive system for masculanizing Aedes aegypti with a scheme for masculanizing A. aegypti by creating a split gene-drive system that masculanizes all (or nearly all) progeny. The top left panel depicts the male genotype in wild-type A. aegypti in which one copy of the first chromosome (or X) carries the M locus (encoded by the Nix gene). Females carry two X-chromosomes lacking the M locus. Middle top panel depicts strain A in which the M locus has been moved from the X-chromosome to a well-defined third chromosome position. The M locus (red) is carried on an element A that also carries gRNA1 (purple), which directs cleavage of the genome at insertion site of element A on the third chromosome, gRNA2 (blue) that cuts the genome at the insertion site of element B, and one or more gRNAs (green) directing cleavage at structurally critical regions of genes encoding proteins required for female fertility (denoted by green Xs in lower panel). Top right panel depicts strain B in which carries a source of Cas9. In strain B, the M locus is in its normal location of the X-chromosome.

The lower panel depicts the result of a cross between a male from strain A with a female from strain B. The resulting progeny lack the M locus on the X chromosome (because strain B females lack this X-linked locus) and instead carry an M-locus transgene on the third chromosome. The Cas9 produced by element B will combine with the gRNAs produced by element A leading to copying of element A to the homologous chromosome (directed by the purple gRNA1), to similar drive of the Cas9 element B (via blue gRNA2), and to the mutagenic disruption of the female fertility loci (green gRNAs, arrows, and Xs).

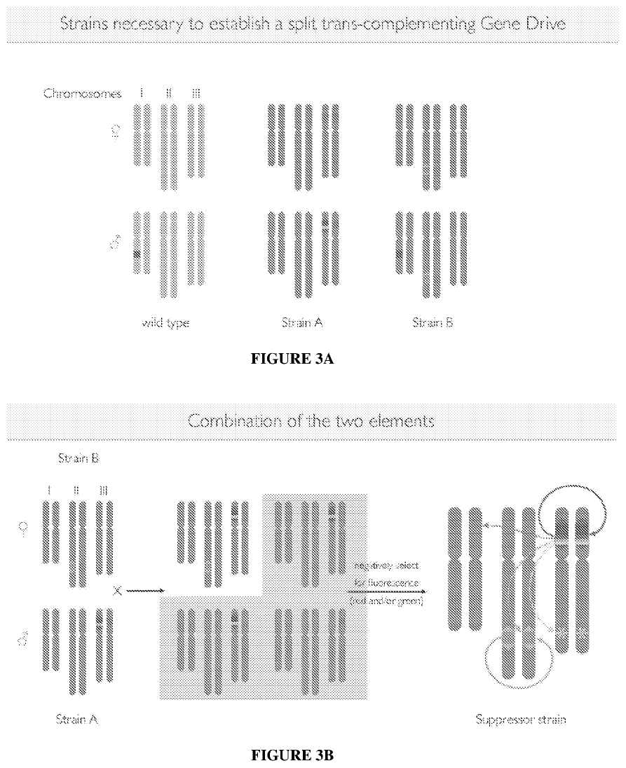

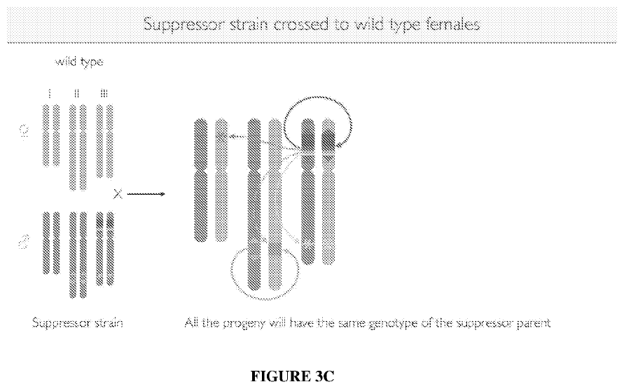

Elements such as A and B behave as standard Mendelian elements until combined after which they behave as a linked gene-drive system referred to as a trans-complementing MCR. When these all male progeny mate with wild-type females, all their offspring should be identical to their fathers, thus leading to a rapid spread of the split trans-complementing gene-drive system via logistical growth. To readily distinguish wild-type from transgenic chromosomes, the transgenic elements A and B can carry distinct fluorescent markers or alternatively, the wild-type chromosomes can be labeled with insertions of fluorescent markers at the same sites at which the A and B elements are inserted, in which case these modified non-driving alleles would serve as local balancers for elements A and B (see FIGS. 3A-B).

As just detailed, this Example provides an embodiment for the development of strains of Ae. aegypti carrying gene-drive constructs capable of suppressing and/or modifying mosquito populations to render them unable to serve as vectors for disease. In particular, provided is a split gene-drive system in which the first element (A) carries the male sex-determining locus (M) at a defined autosomal location (e.g., kynurenine hydroxylase [kh] locus on third chromosome) such that it can be driven by a separate Cas9 source (element B) (FIG. 1). In the strain carrying element A, the endogenous male determining locus on the first-chromosome (or X chromosome) is segregated away resulting in transplantation of the M locus to a precisely defined autosomal site. The A element also carries several guide RNAs (gRNAs) that target Cas9 mediated drive at its own insertion site as well as mutating coding sequences of several genes required for female fertility.

The B element carries Cas9 but does not contain a gRNA. When strains A and B are crossed, however, (males of A crossed to females of B) the Cas9 carried by element B not only drives copying of element B to the homologous chromosome, but also drives copying of element A, resulting in all progeny of the cross being male. This male-drive also occurs at each subsequent generation resulting in the eventual conversion of the entire mosquito population into males. The few non-converted females that might occasionally arise due to errors in copying of element A (e.g., alleles at the M locus generated by non-homologous end joining) are then rendered sterile by the gRNAs carried on element A that target coding regions of loci required for female fertility.

Further provided in this Example is the development of the population modification gene-drive system in which a male germline-specific source of Cas9 drives copying of an effector cassette preventing virus propagation (e.g., Dengue, Zika, Chikungunya). This then is coupled (reduce and replace) with several gRNAs targeting female fertility loci. This latter approach modifies the rare individuals that escape the effects of the suppression drive to render them incapable of propagating viral pathogens. The effector cassettes include combinations of anti-sense RNAs targeting viral RNAs for degradation or hammerhead RNAs. One embodiment of the Example includes the validated anti-dengue-RNAi gene cassette on the suppression gene-drive element. Additional modification effectors as anti-viral gene cassettes can be provided on gRNA-drive elements. Ultimately, combinations of effectors can be added to prevent replication of all viral variants of concern, and such effectors can be combined on the suppression drive element to create a comprehensive suppression/modification drive system.

### Example 2

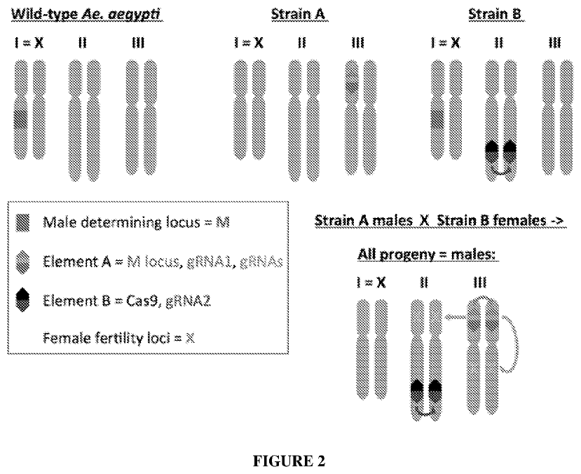

FIG. 2 illustrates a split gene-drive system for masculanizing Aedes aegypti with a second scheme for masculanizing A. aegypti by creating a split gene-drive masculanizing system. Top left panel depicts the male genotype in wild-type A. aegypti in which one copy of the first chromosome (or X) carries the M locus (encoded by the Nix gene). Females carry two X-chromosomes lacking the M locus. Middle top panel depicts strain A in which the M locus has been moved from the X-chromosome to a well-defined third chromosome position. The M locus (red) is carried on an element A that also carries gRNA1 (purple), which directs cleavage of the genome at insertion site of element A on the third chromosome as well as gRNAs (green) directing cleavage at structurally critical regions of genes encoding proteins required for female fertility (denoted by green Xs in lower panel). Top right panel depicts strain B in which carries an active gene-drive element B (also referred to as an MCR element) that carries both a source of Cas9 and gRNA2 (blue) that cuts the genome at its insertion site. In strain B, the M locus is in its normal location of the X-chromosome.

The lower panel depicts the result of a cross between a male from strain A with a female from strain B. The resulting progeny lack the M locus on the X chromosome (because strain B females do not have this X-linked locus) and instead carry an M-locus transgene on the third chromosome. Under idealized conditions all progeny will inherit the gene-drive element B. The Cas9 produced by element B will also combine with the gRNAs produced by element A leading to copying of element A to the homologous chromosome (directed by the purple gRNA) and to the mutagenic disruption of the female fertility loci (green arrow and Xs).

### Example 3

Elements such as A that can copy themselves to the homologous chromosome in the presence of a Cas9 source are referred to as CopyCat elements. When these all male progeny mate with wild-type females, their offspring will all be identical to their fathers, thus leading to a rapid spread of the split gene-drive system via logistical growth. To readily distinguish wild-type from transgenic chromosomes the transgenic elements A and B can also carry distinct fluorescent markers or alternatively, the wild-type chromosomes can be labeled with insertions of fluorescent markers at the same sites at which the A and B elements are inserted, in which case these modified non-driving alleles would serve as local balancers for elements A and B (see FIGS. 3A-B). FIGS. 3A-C show strains useful to establish a split trans-complementing gene drive with a combination of two elements and a suppressor strain crossed to wild type females.

### REFERENCES

1. Gantz, V. and E. Bier. The mutagenic chain reaction: a method for converting heterozygous to homozygous mutations. 2015. Science 348, 442-4; Epub 3/19/15 ScienceExpress DOI:10.1126/science.aaa5945.
2. Gantz V., N. Jasinskiene, O. Tatarenkova, A. Fazekas, V. M. Macias, E. Bier*, and A. A. James*. (2015). Highly efficient Cas9-mediated gene drive for population modification of the malaria vector mosquito, Anopheles stephensi. Proc Natl Acad Sci, In Press. *Co-corresponding authors.
3. Gantz, V., and E Bier. (2016). The dawn of active genetics. BioEssays 38, 50-63; Epub 12/10/15 DOI: 10.1002/bies.201500102.
4. Hall A. B., Basu S., Jiang X., Qi Y. Timoshevskiy V. A., Biedler J. K., Sharakhova M. V., Elahi R., Anderson M. A. E. Chen X.-G., Sharakhov I. V., Adelman Z. N., and Tu Z. (2015). A male-determining factor in the mosquito Aedes aegypti. Science 348, 1268-70.
5. PCT Application No. PCT/US2015/058961 entitled: METHODS FOR AUTOCATALYTIC GENOME EDITING AND NEUTRALIZING AUTOCATALYTIC GENOME EDITING.

1. A genetically modified Aedes aegypti having a Cas9-mediated split gene-drive system for masculinizing the Aedes aegypti.

2. The genetically modified Aedes aegypti of claim 1, wherein the Cas9-mediated split gene-drive system also causes mutations to fertility loci.

3. The genetically modified Aedes aegypti of claim 1, wherein the Cas9-mediated split gene-drive system also causes mutations to insecticide-resistance loci.

4. The genetically modified Aedes aegypti of claim 1, wherein the Cas9-mediated split gene-drive system includes at least one anti-viral effector cassette.

5. The method of claim 4, wherein the at least one anti-viral effector cassette encodes an anti-viral RNAi.

6. The genetically modified Aedes aegypti of claim 1, wherein the Cas9-mediated split gene-drive system comprises a first genetic element and a second genetic element.

7. The genetically modified Aedes aegypti of claim 6, wherein the first genetic element carries the male sex-determining locus (M) at a defined autosomal location such that it can be driven by a Cas9 source provided in trans.

8. The genetically modified Aedes aegypti of claim 7, wherein the defined autosomal location is a kh locus on a third chromosome.

9. The genetically modified Aedes aegypti of claim 7, wherein the defined autosomal location is inside a coding region of a gene required for spermatogenesis.

10. The genetically modified Aedes aegypti of claim 7, wherein an endogenous male-determining locus on a first chromosome is segregated away.

11. A method of suppressing a wild-type population of Aedes aegypti comprising breeding the wild-type population with a genetically modified Aedes aegypti population having a Cas9-mediated split gene-drive system for masculinizing the Aedes aegypti of claim 1.

12. A method of producing a genetically modified Aedes aegypti comprising introducing into the Aedes aegypti a Cas9-mediated split gene-drive system for masculinizing the Aedes aegypti.

13. The method of claim 12, wherein the Cas9-mediated split gene-drive system is introduced by crossing a strain A carrying a genetic element A with a strain B, carrying a genetic element B, wherein strain A does not have a male sex-determining locus at an endogenous location, and wherein the genetic element A comprises the male sex-determining locus.

14. The method of claim 13, wherein the genetic element A encodes: 1) a gRNA driving the genetic element A at its insertion site, 2) a gRNA driving the genetic element B at its insertion site, and 3) multiple gRNAs targeting coding sequences of several genes required for female fertility for mutagenesis through non-homologous end joining,

15. The method of claim 14, wherein the genetic element B encodes a Cas9 endonuclease.

16. The method of claim 15, wherein when males of strain A are crossed to females of strain B, an expressed Cas9 endonuclease encoded by element B drives copying of the genetic elements A and B onto identical insertion sites at their respective sister chromosomes and sequence mutations at sites targeted by the multiple gRNAs targeting coding sequences of several genes required for female fertility.

17. The method of claim 16, wherein progeny carrying both of the genetic elements A and B are male, and transmit the masculanizing trait on to 95% or more of the progeny's progeny and subsequent generations.

18. The method of claim 13, wherein progeny resulting from crossing the strain A with the strain B are sterile females.

19. The method of claim 13, wherein the genetic element B encodes a Cas9 endonuclease and a gRNA driving the genetic element B at its insertion site, thereby creating a full gene drive at the locus for genetic element B.

20. The method of claim 13, wherein the genetic elements A and B or corresponding genomic insertion sites on wild-type chromosomes further comprise a fluorescent marker gene to distinguish transgenic from wild-type chromosomes.
